(19) United States
(12) Patent Application Publication
(10) Pub. No.: US 2026/0234587 A1
(43) Pub. Date: Aug. 13, 2026

(54) Enhanced CAS9 Nickase with Improved Activity

(71) Applicant: Integrated DNA Technologies, Inc., Coralville, IA (US)

(72) Inventors: Michael Allen COLLINGWOOD, Solon, IA (US); Sarah Franz BEAUDOIN, Iowa City, IA (US); Jing LI, Coralville, IA (US); Christopher Anthony VAKULSKAS, North Liberty, IA (US)

(21) Appl. No.: 19/422,665

(22) Filed: Dec. 17, 2025

Related U.S. Application Data
(60) Provisional application No. 63/735,458, filed on Dec. 18, 2024.

(51) Int. Cl. C12N 9/22; C12N 15/11; C12N 15/90

(52) U.S. Cl. CPC C12N 9/226; C12N 15/11; C12N 15/902; C12N 2310/20

(57) ABSTRACT

Provided herein are Cas9 nickase mutations with enhanced prime editing activity relative to wild type for use in prime editing using a prime editing guide RNA.

This application claims the benefit of U.S. Ser. No. 63/735,458, filed Dec. 18, 2024, the entirety of which is incorporated herein by reference.

### REFERENCE TO AN ELECTRONIC SEQUENCE LISTING

The contents of the electronic sequence listing, 6391-0024PV01_12152024.xml; Size: 24,511 bytes; and Date of Creation: Dec. 15, 2024, is herein incorporated by reference in its entirety.

### BACKGROUND

This disclosure pertains to the ability of a nickase CRISPR/Cas9 mutant to cleave double-stranded DNA on one strand in a targeted manner in living cells when complexed with sgRNAs. SpCas9 is an RNA guided endonuclease from the Clustered Regularly Interspaced Short Palindromic Repeat (CRISPR)-Cas (CRISPR-associated) bacterial adaptive immune system of Streptococcus pyogenes [1]. Cas9 is guided to a 23-nt DNA target sequence by a target site-specific 20-nt complementary RNA (part of the 44-nt crRNA) and a universal 89-nt tracrRNA, collectively referred to as the guide RNA (gRNA) complex. The Cas9-gRNA ribonucleoprotein (RNP) complex mediates double-stranded DNA breaks (DSBs) which are then typically repaired by the non-homologous end joining (NHEJ), microhomology mediated end joining (MMEJ), or homology-directed repair (HDR) system if a suitable template nucleic acid is present.

S. pyogenes Cas9 protein contains two endonuclease domains that function together to generate a double-strand DNA break by cleaving both the target (guide complementary) and non-target (guide noncomplementary) strands of a double-stranded DNA (dsDNA). These conserved domains are the RuvC and HNH domains. There are two known mutations that can alter Cas9, which produces double-stranded cleavage, into a ‘nickase’ that results in single-stranded cleavage. Cas9 D10A variant, in the RuvC domain, generates the nick on the targeted strand, while the Cas9 H840A variant, in the HNH domain, generates the nick on the non-targeted strand [1]. The nickase Cas9 variants have been used to facilitate CRISPR-targeted genome editing approaches that do not rely on the introduction of a dsDNA break, examples of which include cytosine/adenine base editors [2,3] and more recently the Cas9 prime editor [4].

Current information suggests that the H840A substitution is not as potent of nickase as the D10A substitution [5], which may indicate that this particular substitution is negatively affecting other aspects of Cas9 function. A mutation that inactivates the HNH non-targeting strand nicking activity without collateral effects on Cas9 function and/or intrinsically works better would enhance and make nickase-based CRISPR genome editing modalities more efficient and effective.

All references cited herein are incorporated herein by reference in their entireties.

### BRIEF SUMMARY

In exemplary embodiments the disclosure provides a modified Cas9 protein, comprising one or more mutations listed in Table 2. The disclosure provides a modified Cas9 protein comprising one or more mutations selected from the group consisting of I830S, I830A, I830G, I830L, I830F, N831A, N831L, N831S, N831K, R832E, R832D, R832H, R832L, R832Q, R832V, L833Q, L833S, L833T, S834P, S834R, V842I, P843T, Q844R, F846Y, D850V, D850E, D850H, and D850Y relative to the wild-type Cas9 amino acid sequence of SEQ ID NO: 23. The disclosure provides a modified Cas9 protein wherein the modified Cas9 protein comprises one or more mutations selected from the group consisting of R832E, R832D, R832H, R832L, I830S, N831A, R832Q, D850Y, D850V, D850E, and F846Y relative to the wild-type Cas9 amino acid sequence of SEQ ID NO: 23. The disclosure provides a modified Cas9 protein wherein the modified Cas9 protein comprises one or more mutations selected from the group consisting of R832E, R832E, D850Y, and D850V relative to the wild-type Cas9 amino acid sequence of SEQ ID NO: 23. The disclosure provides a modified Cas9 protein comprising one or more mutations selected from the group consisting of A840Y, A840L, A840F, A840W, A840M, A840C, A840E, A840R, A840S, A840N, A840T, A840D, A840Q, A840K, A840V, A840I, A840G, and A840P relative to a control Cas9 nickase amino acid sequence of SEQ ID NO: 22. The disclosure provides a modified Cas9 protein wherein the modified Cas9 protein comprises one or more mutations selected from the group consisting of A840Y, A840L, A840F, A840W, A840M, A840C, A840E, A840R, A840S, A840N, and A840T relative to a control Cas9 nickase amino acid sequence of SEQ ID NO: 22. The disclosure provides a modified Cas9 protein wherein the modified Cas9 protein comprises one or more mutations selected from the group consisting of A840Y, A840L, A840F, A840W, and A840M relative to a control Cas9 nickase amino acid sequence of SEQ ID NO: 22. The disclosure provides a modified Cas9 protein further comprising at least one nuclear localization signal. The disclosure provides a modified Cas9 protein wherein the modified Cas9 protein exhibits enhanced nickase activity relative to the wild-type Cas9 having the sequence of SEQ ID NO: 23 or the control Cas9 nickase having the sequence of SEQ ID NO: 22. The disclosure provides a modified Cas9 protein wherein the modified Cas9 protein comprises one or more mutations that confer increased nickase activity relative to the control Cas9 nickase having the sequence of SEQ ID NO: 22. The disclosure provides a modified Cas9 protein wherein the modified Cas9 protein binds to a pegRNA and exhibits prime editing activity enhanced by at least an amount selected from the group consisting of 5%, 10%, 15%, 20%, 25%, and 30%, relative to the control Cas9 nickase having the sequence of SEQ ID NO: 22. The disclosure provides an isolated nucleic acid encoding the modified Cas9 protein as disclosed herein. The disclosure provides an mRNA encoding the modified Cas9 protein as disclosed herein.

The disclosure provides a method for isolating a modified Cas9 protein as disclosed herein, the method comprising: (a) generating a library of Cas9 protein variants through saturation mutagenesis of one or more target codons within a nucleic acid sequence encoding Cas9 protein; (b) cloning the mutated nucleic acid encoding Cas9 protein along with a nucleic acid encoding a pegRNA which targets an antibiotic resistance gene into the same or different plasmid vector; (c) introducing the plasmid vector(s) into a host cell population with a separate plasmid encoding a deactivated antibiotic resistance gene; (d) subjecting the host cell population to antibiotic selection; and (e) isolating modified Cas9 protein variants that exhibit enhanced prime editing activity with pegRNA to restore antibiotic resistance, thereby isolating a modified Cas9 protein that functions with a prime editing guide RNA (pegRNA) that exhibits enhanced prime editing activity relative to the wild-type Cas9 having the sequence of SEQ ID NO: 23 or the control Cas9 nickase having the sequence of SEQ ID NO: 22.

The disclosure provides a method for isolating a modified Cas9 protein as disclosed herein wherein the target codons within the nucleic acid sequence encoding Cas9 nickase encode amino acids at a position selected from the group consisting of 830, 831, 832, 833, 834, 835, 836, 837, 838, 839, 840, 841, 842, 843, 844, 845, 846, 847, 848, 849 and 850.

The disclosure provides a method for isolating a modified Cas9 protein as disclosed herein wherein the host cell is selected from the group consisting of bacterial cells, insect cells, or mammalian cells.

The disclosure provides a method for isolating a modified Cas9 protein as disclosed herein wherein the modified Cas9 nickase that functions with the pegRNA exhibits prime editing activity enhanced by at least an amount selected from the group consisting of 5%, 10%, 15%, 20%, 25%, and 30%, relative to the wild-type Cas9 having the sequence of SEQ ID NO: 23 or the control Cas9 nickase having the sequence of SEQ ID NO: 22.

The disclosure provides a method for isolating a modified Cas9 protein as disclosed herein wherein the saturation mutagenesis targets specific codons within a Cas9 nickase domain selected from the group consisting of the REC Lobe; the Bridge Helix; the NUC Lobe; the RuvC Domain; the HNH Domain; the PAM-Interacting Domain; and the C Terminal Domain.

The disclosure provides a method for isolating a modified Cas9 protein as disclosed herein wherein the mutations are located in a Cas9 nickase domain selected from the group consisting of REC1, REC2, REC3, and combinations thereof.

The disclosure provides a method for isolating a modified Cas9 protein as disclosed herein wherein the pegRNA targets a kanamycin resistance gene.

The disclosure provides a method for isolating a modified Cas9 protein as disclosed herein wherein the bacterial host cell expresses a reporter gene under the control of a promoter responsive to Cas9 nickase activity, wherein expression of the antibiotic resistance gene indicates enhanced potency of the modified Cas9 protein.

The disclosure provides a modified Cas9 protein as disclosed herein wherein the modified Cas9 protein functions with a pegRNA that exhibits enhanced prime editing activity relative to the control Cas9 nickase having the sequence of SEQ ID NO: 22, wherein the modified Cas9 protein comprises one or more mutations that confer increased potency relative to the control Cas9 nickase.

The disclosure provides a modified Cas9 protein as disclosed herein wherein the modified Cas9 protein that functions with a pegRNA exhibits prime editing activity enhanced by at least an amount selected from the group consisting of 5%, 10%, 15%, 20%, 25%, and 30%, relative to the control Cas9 nickase having the sequence of SEQ ID NO: 22.

The disclosure provides a host cell comprising a nucleic acid encoding the modified Cas9 protein as disclosed herein.

The disclosure provides a composition comprising the modified Cas9 protein as disclosed herein formulated for use in biochemical assays, industrial processes, or therapeutic applications.

The disclosure provides a gene editing system, comprising: a. at least one of the modified Cas9 protein as disclosed herein; and b. at least one of a guide RNA (gRNA) or a pegRNA, wherein the gene editing system exhibits enhanced editing activity relative to the wild-type Cas9 having the sequence of SEQ ID NO: 23 or the control Cas9 nickase having the sequence of SEQ ID NO: 22.

The disclosure provides a method of delivering a gene editing system as disclosed herein to a cell, the method comprising the steps of: (a) providing a first viral vector component encoding a pegRNA that hybridizes with a target sequence; (b) providing a second viral vector component encoding a modified Cas9 protein as disclosed herein; wherein components (a) and (b) are located on same or different vectors of the system; and (c) transducing the cell with the viral vector(s) under conditions sufficient to express the modified Cas9 protein and the pegRNA, wherein the Cas9 nickase and the pegRNA form a complex that binds to and edits the target sequence.

The disclosure provides a method of delivering a gene editing system as disclosed herein to a cell, comprising: (a) providing lipid nanoparticles encapsulating a pegRNA that hybridizes with a target sequence; (b) providing lipid nanoparticles encapsulating an mRNA encoding a modified Cas9 protein as disclosed herein; wherein components (a) and (b) are located on same or different vectors of the system, (c) transducing the cell with the lipid nanoparticles under conditions sufficient to express the modified Cas9 protein and the pegRNA, wherein the modified Cas9 protein and the pegRNA are expressed and form a complex to edit a specific target sequence in the cell's genome.

The disclosure provides a method of performing gene editing having reduced off-target editing activity and/or increased on-target editing activity, comprising: (a) providing a ribonucleoprotein (RNP) complex comprising the gene editing system as disclosed herein; and (b) introducing the RNP complex into the cell using electroporation, wherein the gene editing system binds to and edits a target sequence within the genome of the cell.

The disclosure provides a method of delivering the gene editing system as disclosed herein to a cell, comprising: preparing a lipofection reagent comprising a first regulatory element operable in a eukaryotic cell operably linked to at least one nucleotide sequence encoding a pegRNA that hybridizes with a target sequence; a second regulatory element operable in a eukaryotic cell operably linked to a nucleotide sequence encoding the modified Cas9 protein; wherein components (a) and (b) are located on the same or different vectors of the system, and applying the lipofection reagent to the cell, wherein the gene editing system are expressed and form a complex to edit a specific target sequence in the cell's genome.

The disclosure provides a method of targeted delivery of the gene editing system as disclosed herein to a cell, comprising: (a) preparing exosomes encapsulating a first nucleotide sequence encoding a pegRNA that hybridizes with a target sequence; and (b) preparing exosomes encapsulating a second nucleotide sequence encoding the modified Cas9 protein, wherein components (a) and (b) are located on same or different vectors of the system; and (c) delivering the engineered exosomes to the cell under conditions that allow the gene editing system to edit the target sequence. The disclosure provides a method of targeted delivery of the gene editing system wherein the cell is selected from the group consisting of bacterial cells, insect cells, plant cells, or mammalian cells.

The disclosure provides a method for producing a modified Cas9 nickase that exhibits enhanced prime editing activity with a pegRNA relative to wild-type Cas9, the method comprising: (a) subjecting a nucleic acid sequence encoding Cas9 nickase to saturation mutagenesis; (b) cloning a mutated nucleic acid encoding Cas9 nickase along with a nucleic acid encoding a pegRNA which targets an antibiotic resistance gene into the same or different plasmid vector; (c) introducing the plasmid vector(s) into a host cell population with a separate plasmid encoding a deactivated antibiotic resistance gene; (d) subjecting the host cell population to antibiotic selection; (e) isolating modified Cas9 nickase variants that exhibit enhanced prime editing activity functional interaction with pegRNA which restores antibiotic resistance, thereby producing a modified Cas9 nickase that functions with a pegRNA that exhibits enhanced prime editing activity relative to wild-type Cas9. The disclosure provides a method for producing a modified Cas9 nickase that exhibits enhanced prime editing activity with a pegRNA relative to wild-type Cas9 wherein the target codons within the nucleic acid sequence encoding Cas9 nickase encode amino acids at a position selected from the group consisting of 830, 831, 832, 833, 834, 835, 836, 837, 838, 839, 840, 841, 842, 843, 844, 845, 846, 847, 848, 849 and 850. The disclosure provides a method for producing a modified Cas9 nickase that exhibits enhanced prime editing activity with a pegRNA relative to wild-type Cas9 wherein the host cell is selected from the group consisting of bacterial cells, insect cells, or mammalian cells. The disclosure provides a method for producing a modified Cas9 nickase that exhibits enhanced prime editing activity with a pegRNA relative to wild-type Cas9 wherein the modified Cas9 nickase that functions with a pegRNA exhibits prime editing activity enhanced by at least an amount selected from the group consisting of 5%, 10%, 15%, 20%, 25%, and 30%, relative to a wild-type Cas9 with a full-length pegRNA. The disclosure provides a method for producing a modified Cas9 nickase that exhibits enhanced prime editing activity with a pegRNA relative to wild-type Cas9 wherein the saturation mutagenesis targets specific codons within a Cas9 nickase domain selected from the group consisting of the REC Lobe; the Bridge Helix; the NUC Lobe; the RuvC Domain; the HNH Domain; the PAM-Interacting Domain; and the C Terminal Domain. The disclosure provides a method for producing a modified Cas9 nickase that exhibits enhanced prime editing activity with a pegRNA relative to wild-type Cas9 wherein the mutations are located in a Cas9 nickase domain selected from the group consisting of REC1, REC2, REC3, and combinations thereof. The disclosure provides a method for producing a modified Cas9 nickase that exhibits enhanced prime editing activity with a pegRNA relative to wild-type Cas9 wherein the pegRNA targets a kanamycin resistance gene. The disclosure provides a method for producing a modified Cas9 nickase that exhibits enhanced prime editing activity with a pegRNA relative to wild-type Cas9 wherein the bacterial host cell expresses a reporter gene under the control of a promoter responsive to Cas9 nickase activity, wherein expression of the antibiotic resistance gene indicates enhanced potency of the Cas9 nickase variant.

### DETAILED DESCRIPTION

The disclosure provides using a bacterial prime editing selection approach to isolate ideal nickase variants to facilitate the highest nickase activity and as a result the most potent prime editing outcome possible. To our knowledge, no comprehensive nickase screen with selection has been performed for either of these domains and the current iterations (D10A and H840A) were isolated through computational structure prediction and testing. Focusing on the H840A variant required for prime editing, we made a mutant library of amino acid substitution in a 21 amino acid window centered around H840. We screened that library for substitutions that would enable prime editing in bacteria with the highest overall potency.

The Cas9 nickase variants, systems, and methods as disclosed herein could be used for any application of a Cas9 non-targeting strand nickase alternative to H840A including paired nickase HDR, base editing, prime editing, or future CRISPR-targeted technologies that rely on ssDNA cleavage. The Cas9 nickase variants, systems, and methods as disclosed herein can be used in live cells or in a cell free context, and can be used as expressed from a virus, plasmid, or mRNA, or alternatively used directly in an RNP as a purified nickase nuclease.

The Clustered Regularly Interspaced Short Palindromic Repeats and CRISPR-associated protein 9 (CRISPR-Cas9) system is a tool in genetic engineering to precisely edit genes within organisms. CRISPR-Cas systems are native to bacteria and Archaea to provide adaptive immunity against viruses and plasmids. CRISPR are sequences of DNA in bacterial genomes that store fragments of viral DNA. If the bacterium survives a viral attack, it incorporates a piece of the virus's genetic material into its own DNA. These stored sequences help the bacteria recognize and destroy a virus or plasmid. Cas9 is an enzyme that cuts DNA at specific sites. It's guided by a guide RNA (gRNA). The gRNA is complementary to the target DNA sequence. This gRNA will guide the Cas9 protein to the exact location in the genome. Once guided to the target location, the Cas9 enzyme cuts the DNA at that precise spot. After Cas9 cuts the DNA, the cell tries to repair the break. Providing a template with the desired DNA sequence can instruct the cell to incorporate this new sequence during the repair process.

There are three classes of CRISPR-Cas systems that could potentially be adapted for research and therapeutic reagents, but Type-II CRISPR systems have a desirable characteristic in utilizing a single CRISPR associated (Cas) nuclease (e.g., Cas9) in a complex with the appropriate guide RNAs—either a 2-part RNA system similar to the natural complex in bacteria comprising a CRISPR-activating RNA: trans-activating crRNA (crRNA: tracrRNA) pair or an artificial chimeric single-guide-RNA (sgRNA)—to mediate double-stranded cleavage of target DNA. In mammalian systems, these RNAs have been introduced by transfection of DNA cassettes containing RNA Pol III promoters (such as U6 or H1) driving RNA transcription, viral vectors, and single-stranded RNA following in vitro transcription (see Xu, T., et al., Appl Environ Microbiol, 2014. 80 (5): p. 1544-52).

Cas9 nickase is a modified version of the Cas9 protein. While the wild-type Cas9 enzyme introduces double-strand breaks (DSBs) in DNA, Cas9 nickase is designed to make a single-strand break, or “nick,” at a specific location on the DNA. This modified version offers more precision and reduces the risk of unintended genomic alterations (off-target effects). Cas9 nickase only cleaves one strand of the DNA, creating a nick rather than a complete break. This happens by mutating one of Cas9's two nuclease domains, either RuvC or HNH, which renders it inactive. As a result, Cas9 retains the ability to cut only one DNA strand. For genome editing, two Cas9 nickases can be used in combination to target opposite strands of DNA close to each other. This creates two single-strand breaks, which can then be treated as a DSB by the cell's repair machinery. DSBs are typically repaired by non-homologous end joining (NHEJ), which is error-prone and can lead to insertions or deletions (indels). Nicks, on the other hand, are primarily repaired through homology-directed repair (HDR), a more accurate repair process if a homologous DNA template is available. The nickase system allows for more precise editing with fewer chances of errors or unwanted mutations, especially when using HDR for precise gene modifications.

Cas9 has two nuclease domains, RuvC and HNH, that are responsible for cleaving the two strands of DNA. To create a nickase, one of these domains is mutated: Mutating the RuvC domain (e.g., D10A mutation) leads to the cutting of the complementary strand only. Mutating the HNH domain (e.g., H840A mutation) results in the nicking of the noncomplementary strand only.

Paired nickases can be used to introduce highly accurate modifications by ensuring both strands are cut only at the intended target sites. Since two nicks are required to create a DSB, the probability of off-target double-strand breaks is significantly reduced compared to wild-type Cas9. Cas9 nickase can be combined with deaminases for base editing, allowing the precise conversion of one base to another (e.g., C to T) without inducing a DSB.

The Cas9 protein consists of several functional regions or domains, each with specific roles in the CRISPR-Cas9 gene-editing process, including the REC Lobe (Recognition Lobe), which contains several subdomains (REC1, REC2, REC3) that are responsible for recognizing and binding the guide RNA (gRNA); the Bridge Helix, which is a helical structure that connects the REC and NUC lobes and plays a role in the allosteric activation of Cas9. The NUC Lobe (Nuclease Lobe) which contains the active nuclease domains and is responsible for cutting the target DNA. The two primary nuclease domains include the RuvC Domain which cleaves the DNA strand that does not pair with the guide RNA (the non-target strand) and the HNH Domain which cleaves the DNA strand that is complementary to the guide RNA (the target strand). The PAM-Interacting Domain (PID) recognizes the protospacer adjacent motif (PAM) sequence, a short DNA sequence adjacent to the target site required for Cas9 binding. Different Cas9 variants recognize different PAM sequences, which determines the target specificity of each Cas9 protein. PAM recognition is a critical step because Cas9 will not bind or cut DNA without the correct PAM sequence. The CTD (C-Terminal Domain) is involved in stabilizing the Cas9-DNA complex.

Prime editing is an advanced genome editing technique derived from CRISPR technology which offers the ability to precisely insert, delete, or alter specific DNA sequences without the need for double-strand breaks (DSBs) or donor DNA templates. In prime editing, the Cas9 nickase is fused to a reverse transcriptase enzyme, which synthesizes a new DNA strand using an RNA template provided by the pegRNA (prime editing guide RNA). This allows for precise edits, including base changes, insertions, or deletions, directly at or near the nicked site. The Prime Editing Guide RNA (pegRNA) is an extended version of the traditional guide RNA (gRNA) which has two main parts: A guide sequence that directs the Cas9 nickase to the correct location in the genome, ensuring the cut happens at the desired site; and a reverse transcriptase template and a primer binding site that encode the desired DNA sequence changes. This template provides the instructions for the reverse transcriptase to make the new DNA sequence, which will replace the original DNA at the target site. Prime editing allows for very precise genome modifications, including base substitutions, small insertions, deletions, and even complex modifications like transversions (e.g., changing a C-G pair to a G-C pair).

The modified Cas9 nickase-pegRNA system components as disclosed herein can be introduced into the cell, together or separately, using various approaches. Examples include plasmid or viral expression vectors (which lead to endogenous expression), Cas9 mRNA with separate pegRNA transfection, or delivery of the mutant Cas9 protein with the pegRNA as a ribonucleoprotein (RNP) complex (see Kouranova et al., Hum Gen Ther (2016) 27 (6): 464-475). Effective strategies for introducing, for example, the Cas9 nickase-pegRNA system as disclosed herein into target cells include, for example, Viral Vectors, such as Adeno-Associated Virus (AAV) which are widely used for CRISPR delivery because they are generally safe, induce minimal immune response, and have been approved in some gene therapy applications; Lentivirus and Retrovirus; and Adenovirus.

Additional methods for introducing the modified Cas9 nickase-pegRNA system as disclosed herein into target cells includes, for example, Lipid Nanoparticles (LNPs) which are commonly used for delivering RNA-based therapies; Electroporation, which involves applying an electrical field to create temporary pores in the cell membrane, allowing CRISPR components (like plasmids, ribonucleoprotein complexes, or mRNA) to enter the cell. Additional methods for introducing the modified Cas9 nickase-pegRNA system as disclosed herein into target cells include, for example, Ribonucleoprotein (RNP) Complexes which involves directly delivering the Cas9 protein pre-complexed with guide RNA (gRNA) into cells, usually via electroporation or lipid-based transfection; Lipid-Based Transfection Agents (lipofection) uses lipid-based reagents to encapsulate CRISPR plasmids or RNP complexes and facilitate their uptake by cells. Other methods for introducing the modified Cas9 nickase-pegRNA system as disclosed herein into target cells include, for example, Physical Methods such as Microinjection to directly injects CRISPR components into cells, typically used in single-cell embryos or zygotes for generating transgenic animals; Nanoneedles and Microfluidics which can introduce CRISPR components with minimal damage to cells; and Exosome-Mediated Delivery, which can be engineered to carry CRISPR/Cas components and target them to specific cells.

Next Generation Sequencing (NGS) allows rapid and high-throughput sequencing of DNA and RNA. Unlike earlier methods such as Sanger sequencing, which sequences one DNA fragment at a time, NGS enables the simultaneous sequencing of millions of DNA fragments, making it much faster, cheaper, and more efficient. In NGS, a DNA or RNA from the sample is extracted and fragmented into smaller pieces. These fragments are then attached to short synthetic DNA sequences called adapters, which are needed for binding to the sequencing platform. The DNA fragments with adapters are amplified (copied many times) to create a “library” of DNA fragments. This increases the amount of DNA available for sequencing. Most NGS platforms, like Illumina, use a method called “sequencing by synthesis.” Each fragment is attached to a solid surface and copied in place. Fluorescently labeled nucleotides (A, T, C, and G) are added one by one. As they bind to the complementary strand, the machine detects the fluorescent signal, allowing the sequence of bases to be read. The massive amount of sequencing data is analyzed using bioinformatics tools. The overlapping DNA fragments are assembled back into their original sequence by aligning them to a reference genome or constructing new genomes (de novo sequencing). NGS is high throughput, since millions to billions of DNA fragments can be sequenced in parallel, producing vast amounts of data, is cost-effective, and can sequence entire genomes or large sets of genes in days, making it much faster than older sequencing methods. Tiled-amplicon NGS is a targeted sequencing method designed to analyze specific regions of a genome or set of genes by amplifying overlapping “tiles” of DNA fragments through polymerase chain reaction (PCR). Each tile covers a portion of the target region, and their overlapping design ensures comprehensive coverage and accurate sequence reconstruction. In tiled-amplicon NGS target regions of interest are divided into overlapping amplicon segments and primers are designed to amplify these amplicons, ensuring overlap between adjacent segments to cover the entire target area. The primers amplify their corresponding tiles using PCR, creating a library of DNA fragments from the target regions. Multiplex PCR is often used, where multiple amplicons are amplified simultaneously in a single reaction. The amplified DNA is prepared for sequencing by adding adapters and barcodes for sample identification and compatibility with the sequencing platform. The prepared DNA library is sequenced on an NGS platform, generating large volumes of short-read sequence data. Sequencing reads are aligned to a reference genome or assembled de novo to reconstruct the target regions. Overlapping tiles help resolve sequencing errors, fill gaps, and increase the accuracy of variant detection.

Saturation mutagenesis is used to introduce mutations at every possible site within a specific region of a gene. This method systematically replaces one or more amino acids in a protein with all other possible amino acids (or nucleotides in a DNA sequence) to explore the effects of these changes on the protein's function. The goal is to understand which mutations enhance or diminish the activity, stability, or other characteristics of a protein, and it is often used in protein engineering. The target selected for saturation mutagenesis can be a specific codon or a set of codons within a protein that are crucial for function. A set of oligonucleotides with randomized nucleotides at the positions where mutations are intended is created. The mutagenized oligonucleotides are incorporated into the target gene via polymerase chain reaction (PCR). The mutagenized gene is then cloned into an expression vector. After introducing the mutated genes into a host organism (such as bacteria), the resulting proteins are expressed. These proteins are then tested for desired properties such as improved enzymatic activity, binding affinity, or stability. The most promising mutants are sequenced to identify which amino acid substitutions are responsible for any observed changes in protein function. By generating a diverse library of mutants, saturation mutagenesis can be used for optimizing protein properties or gaining deeper insight into protein function.

A variety of host cells can serve as platforms for CRISPR expression, such as: Bacterial Cells: Escherichia coli is widely used for CRISPR applications like plasmid construction, cloning, and CRISPR screens; Yeast: Saccharomyces cerevisiae and other yeast species can be genetically modified to express CRISPR systems, especially in studies focused on gene function and genome screening in eukaryotic systems; Insect cells can serve as hosts for CRISPR expression. In particular, insect cell lines such as Sf9 (from Spodoptera frugiperda) and S2 (from Drosophila melanogaster); Mammalian Cells: Various mammalian cell lines, including HEK293, HeLa, and CHO cells, are commonly used, which are compatible with more complex CRISPR modifications, such as large gene insertions, knock-ins, or base editing, due to their complex regulatory machinery; Primary Cells and Stem Cells: Primary cells, like human or animal-derived cells, and induced pluripotent stem cells (iPSCs) can also be used as host cells for CRISPR systems, especially for therapeutic studies and disease modeling; Plant Cells: Plants like Arabidopsis thaliana, tobacco, and rice can serve as CRISPR host cells. Plant cells are often transformed with CRISPR machinery to study gene function, enhance traits, or improve resistance to pathogens. Various delivery systems for CRISPR components, such as plasmids, viral vectors, or ribonucleoprotein complexes, and specific promoters can be optimized for the host's transcriptional machinery.

The term “nucleic acid” refers to a nucleotide polymer, and unless otherwise limited, includes analogs of natural nucleotides that can function in a similar manner (e.g., hybridize) to naturally occurring nucleotides. The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acids, such as nucleic acid templates. Methods described herein additionally provide for the generation of isolated and at least partially purified nucleic acids and libraries of nucleic acids. Nucleic acids include but are not limited to those comprising DNA, RNA, circular RNA, cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like. More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs).

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to the capacity for precise pairing between two nucleotides, i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules. The term “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base (a single nucleotide is also referred to as a “base” or “residue”). There is no intended distinction in length between the terms “nucleic acid”, “oligonucleotide” and “polynucleotide”, and these terms can be used interchangeably. These terms refer only to the primary structure of the molecule. Thus, these terms include double- and single-stranded DNA, as well as double- and single-stranded RNA. For use in the system and methods as disclosed herein, an oligonucleotide also can comprise nucleotide analogs in which the base, sugar or phosphate backbone is modified as well as non-purine or non-pyrimidine nucleotide analogs. An oligonucleotide may comprise ribonucleotides, deoxyribonucleotides, modified nucleotides (e.g., nucleotides with 2′ modifications, synthetic base analogs, etc.) or combinations thereof.

The term “ribonucleotide” encompasses natural and synthetic, unmodified and modified ribonucleotides. Modifications include changes to the sugar moiety, to the base moiety and/or to the linkages between ribonucleotides in the oligonucleotide.

The term “polypeptide” refers to any linear or branched peptide comprising more than one amino acid. Polypeptide includes protein or fragment thereof or fusion thereof, provided such protein, fragment or fusion retains a useful biochemical or biological activity.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example, “1, 2 and/or 3” is equivalent to “1, 2, 3, 1 and 2, 1 and 3, 2 and 3, or 1, 2, and 3”.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The disclosure as illustratively described herein suitably may be practiced in the absence of any element, elements, limitation, or limitations which is not specifically disclosed herein.

As used herein and in the appended claims, the singular forms “a”, “an”, and “the” include plural referents unless the context clearly dictates otherwise. Thus, for example, reference to “a method” includes a plurality of such methods and reference to “the nanoparticle” includes reference to one or more nanoparticles and equivalents thereof known to those skilled in the art, and so forth. The terms and expressions which have been employed are used as terms of description and not of limitation, and there is no intention in the use of such terms and expressions of excluding any equivalents of the features shown and described or portions thereof, but it is recognized that various modifications are possible within the scope as disclosed herein claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references throughout this application, for example patent documents, including issued or granted patents or equivalents and patent application publications, and non-patent literature documents or other source material are hereby incorporated by reference herein in their entireties, as though individually incorporated by reference. None is admitted to be prior art.

As used herein, the term “about” when used in conjunction with a stated numerical value or range has the meaning reasonably ascribed to it by a person skilled in the art, i.e., denoting somewhat more or somewhat less than the stated value or range.

The invention will be illustrated in more detail with reference to the following Examples, but it should be understood that the present invention is not deemed to be limited thereto.

### EXAMPLES

### Example 1—a Bacterial Prime Editing Selection and Enrichment Strategy Reveals Nickase Mutations that Facilitate the Most Potent Prime Editor

Functional prime editing has recently been described in E. coli using a variety of insertions/deletions and substitutions [6]. We used expression of a prime editor and PEG-RNA that targets a kanamycin resistance gene that contains a 10-base disruption between codons 1 and 2. This prime editing strategy is designed to remove the disrupting 10-base element and restore kanamycin resistance gene expression, and thus confer resistance to the antibiotic kanamycin.

The screen is setup such that a library of Cas9 Prime Editor mutants and PEG RNA are expressed from one plasmid, and the target site and non-functional kanamycin resistance gene is present on a second plasmid. Practically, bacterial cells that stably replicate the target site-containing plasmid are made competent, transformed using Cas9 prime editor and PEG RNA plasmid, and selected on Kanamycin-containing solid media.

A saturation mutagenesis library (Table 1) within Cas9 amino acids 830-850 was generated with nicking mutagenesis and the resulting library complexity was analyzed with tiled-amplicon NGS. This library was delivered into E. coli cells as described above to be greater than 99% confident that each possible codon change was observed at least once. The resulting colonies were pooled, plasmids from this pool were purified, and the resulting pool was sequenced with overlapping tiled amplicon NGS. Pre- and post-enrichment pools were sequenced and compared simultaneously with total read count normalization to determine enrichment for each substitution within the pool. Paired end reads were merged by fastp [7], trimmed to keep the desired mutated region by Cutadapt [8], and filtered out undesired reads with wrong length or multiple codon mutation. Codon frequency and enrichment analysis were completed by in-house python script. The results indicate that the H840 position is the most frequently isolated mutation, but that an alanine substitution is far from the most frequently observed mutation indicating that it is not the most ideal prime editing nickase (Table 2). Exemplary Cas9 amino acid sequences are set forth in Table 3.

TABLE 1
Primers used for saturation mutagenesis of the desired nickase region.

| | Sequence (5′-3′) |
|---|---|
| SEQ ID NO: 1 | /5Phos/TATGTACGTGGACCAAGAGTTGGATNNKAATCGGCTGAGTGACTACGACGTTG |
| SEQ ID NO: 2 | /5Phos/GTACGTGGACCAAGAGTTGGATATTNNKCGGCTGAGTGACTACGACGTTGATC |
| SEQ ID NO: 3 | /5Phos/CGTGGACCAAGAGTTGGATATTAATNNKCTGAGTGACTACGACGTTGATCATA |
| SEQ ID NO: 4 | /5Phos/GGACCAAGAGTTGGATATTAATCGGNNKAGTGACTACGACGTTGATCATATCG |
| SEQ ID NO: 5 | /5Phos/CCAAGAGTTGGATATTAATCGGCTGNNKGACTACGACGTTGATCATATCGTCC |
| SEQ ID NO: 6 | /5Phos/AGAGTTGGATATTAATCGGCTGAGTNNKTACGACGTTGATCATATCGTCCCGC |
| SEQ ID NO: 7 | /5Phos/GTTGGATATTAATCGGCTGAGTGACNNKGACGTTGATCATATCGTCCCGCAGA |
| SEQ ID NO: 8 | /5Phos/GGATATTAATCGGCTGAGTGACTACNNKGTTGATCATATCGTCCCGCAGAGCT |
| SEQ ID NO: 9 | /5Phos/TATTAATCGGCTGAGTGACTACGACNNKGATCATATCGTCCCGCAGAGCTTCC |
| SEQ ID NO: 10 | /5Phos/TAATCGGCTGAGTGACTACGACGTTNNKCATATCGTCCCGCAGAGCTTCCTCA |
| SEQ ID NO: 11 | /5Phos/TCGGCTGAGTGACTACGACGTTGATNNKATCGTCCCGCAGAGCTTCCTCAAAG |
| SEQ ID NO: 12 | /5Phos/GCTGAGTGACTACGACGTTGATCATNNKGTCCCGCAGAGCTTCCTCAAAGACG |
| SEQ ID NO: 13 | /5Phos/GAGTGACTACGACGTTGATCATATCNNKCCGCAGAGCTTCCTCAAAGACGATT |
| SEQ ID NO: 14 | /5Phos/TGACTACGACGTTGATCATATCGTCNNKCAGAGCTTCCTCAAAGACGATTCTA |
| SEQ ID NO: 15 | /5Phos/CTACGACGTTGATCATATCGTCCCGNNKAGCTTCCTCAAAGACGATTCTATTG |
| SEQ ID NO: 16 | /5Phos/CGACGTTGATCATATCGTCCCGCAGNNKTTCCTCAAAGACGATTCTATTGACA |
| SEQ ID NO: 17 | /5Phos/CGTTGATCATATCGTCCCGCAGAGCNNKCTCAAAGACGATTCTATTGACAATA |
| SEQ ID NO: 18 | /5Phos/TGATCATATCGTCCCGCAGAGCTTCNNKAAAGACGATTCTATTGACAATAAGG |
| SEQ ID NO: 19 | /5Phos/TCATATCGTCCCGCAGAGCTTCCTCNNKGACGATTCTATTGACAATAAGGTAC |
| SEQ ID NO: 20 | /5Phos/TATCGTCCCGCAGAGCTTCCTCAAANNKGATTCTATTGACAATAAGGTACTGA |
| SEQ ID NO: 21 | /5Phos/CGTCCCGCAGAGCTTCCTCAAAGACNNKTCTATTGACAATAAGGTACTGACGC |

TABLE 2
Top amino acid changes that show potential benefit in Prime
Editing application. NGS reads were analyzed against pre-
and post-selection libraries and denoted as fold
change.

| | | | | Post- | |
|---|---|---|---|---|---|
| | | | | Selection | |
| | | | | Normalized | |
| | | Pre- | Post- | to Pre- | |
| Amino Acid | AA Codon | Selection | Selection | Selection | Fold |
| Position | Change | Reads | Reads | Reads | Change |
| R832E | R832E_CGG_GAG | 511 | 3762 | 4914 | 9.62 |
| R832D | R832D_CGG_GAT | 223 | 901 | 1177 | 5.28 |
| R832H | R832H_CGG_CAT | 279 | 724 | 946 | 3.39 |
| R832L | R832L_CGG_CTT | 490 | 1058 | 1382 | 2.82 |
| I830S | I830S_ATT_TCT | 384 | 787 | 1028 | 2.68 |
| N831A | N831A_AAT_GCG | 4001 | 7734 | 10102 | 2.52 |
| R832Q | R832Q_CGG_CAG | 1655 | 2958 | 3864 | 2.33 |
| I830A | I830A_ATT_GCG | 610 | 905 | 1182 | 1.94 |
| N831L | N831L AAT CTG | 3097 | 4269 | 5576 | 1.80 |
| R832L | R832L_CGG_CTG | 5645 | 7676 | 10026 | 1.78 |
| S834P | S834P_AGT_CCT | 1474 | 1999 | 2611 | 1.77 |
| R832L | R832L_CGG_TTG | 833 | 1106 | 1445 | 1.73 |
| L833Q | L833Q_CTG_CAG | 2754 | 2869 | 3747 | 1.36 |
| N831A | N831A_AAT_GCA | 1 | 1 | 1 | 1.31 |
| N831S | N831S AAT TCC | 1 | 1 | 1 | 1.31 |
| I830G | I830G ATT GGT | 1279 | 1200 | 1567 | 1.23 |
| R832V | R832V_CGG_GTG | 1328 | 1197 | 1564 | 1.18 |
| S834R | S834R_AGT_AGA | 873 | 766 | 1001 | 1.15 |
| I830L | I830L ATT_TTA | 6 | 5 | 7 | 1.09 |
| L833S | L833S_CTG_TCG | 1878 | 1562 | 2040 | 1.09 |
| L833T | L833T_CTG_ACG | 1764 | 1465 | 1914 | 1.08 |
| I830F | I830F_ATT_TTT | 3237 | 2626 | 3430 | 1.06 |
| N831K | N831K AAT AAA | 535 | 411 | 537 | 1.00 |
| A840Y | A840Y_GCG_TAT | 3234 | 74301 | 71271 | 22.04 |
| A840L | A840L_GCG_CTG | 2615 | 47213 | 45287 | 17.32 |
| A840F | A840F_GCG_TTT | 3191 | 51853 | 49738 | 15.59 |
| A840L | A840L_GCG_TTG | 3803 | 61144 | 58650 | 15.42 |
| A840L | A840L_GCG_CTT | 2405 | 33695 | 32321 | 13.44 |
| A840W | A840W_GCG_TGG | 4947 | 60385 | 57922 | 11.71 |
| A840M | A840M_GCG ATG | 2651 | 31778 | 30482 | 11.50 |
| A840C | A840C_GCG_TGT | 4527 | 42373 | 40645 | 8.98 |
| A840E | A840E_GCG_GAG | 3431 | 30141 | 28912 | 8.43 |
| A840R | A840R_GCG_CGT | 2900 | 24069 | 23087 | 7.96 |
| A840R | A840R_GCG AGG | 3619 | 29870 | 28652 | 7.92 |
| A840S | A840S_GCG_AGT | 3381 | 23973 | 22995 | 6.80 |
| A840S | A840S_GCG_TCT | 1857 | 13122 | 12587 | 6.78 |
| A840R | A840R GCG CGG | 3473 | 24372 | 23378 | 6.73 |
| A840S | A840S_GCG_TCG | 2393 | 16211 | 15550 | 6.50 |
| A840N | A840N_GCG_AAT | 2597 | 16318 | 15652 | 6.03 |
| A840T | A840T_GCG ACG | 1980 | 11534 | 11064 | 5.59 |
| A840A | A840A GCG GCT | 2608 | 15181 | 14562 | 5.58 |
| A840E | A840E_GCG GAA | 3 | 17 | 16 | 5.44 |
| D850Y | D850Y_GAT TAT | 3 | 17 | 16 | 5.44 |
| A840D | A840D_GCG_GAT | 4185 | 22289 | 21380 | 5.11 |
| A840T | A840T_GCG_ACT | 1528 | 7720 | 7405 | 4.85 |
| D850V | D850V_GAT GTG | 2 | 10 | 10 | 4.80 |
| A840A | A840A_GCG_GCG | 2937 | 14115 | 13539 | 4.61 |
| A840Q | A840Q_GCG_CAG | 2240 | 10230 | 9813 | 4.38 |
| A840L | A840L_GCG_TTA | 6 | 27 | 26 | 4.32 |
| A840K | A840K_GCG_AAA | 7 | 29 | 28 | 3.97 |
| A840K | A840K_GCG AAG | 2598 | 10726 | 10289 | 3.96 |
| A840V | A840V_GCG_GTT | 4049 | 15487 | 14855 | 3.67 |
| A840I | A840I_GCG_ATT | 2831 | 10672 | 10237 | 3.62 |
| A840G | A840G_GCG_GGA | 2 | 7 | 7 | 3.36 |
| A840V | A840V_GCG_GTG | 4428 | 14864 | 14258 | 3.22 |
| A840P | A840P_GCG_CCT | 1470 | 4855 | 4657 | 3.17 |
| D850E | D850E_GAT_GAA | 5 | 16 | 15 | 3.07 |
| A840P | A840P_GCG_CCG | 1725 | 5343 | 5125 | 2.97 |
| F846Y | F846Y_TTC_TAC | 2 | 5 | 5 | 2.40 |
| A840L | A840L_GCG_CTA | 9 | 19 | 18 | 2.03 |
| L833Q | L833Q_CTG_CAG | 3 | 6 | 6 | 1.92 |
| V842I | V842I_GTC_ATC | 2 | 4 | 4 | 1.92 |
| D850H | D850H GAT CAT | 2 | 4 | 4 | 1.92 |
| A840G | A840G_GCG GGT | 5339 | 9094 | 8723 | 1.63 |
| P843T | P843T_CCG_ACG | 2 | 3 | 3 | 1.44 |
| Q844R | Q844R_CAG_CGG | 2 | 3 | 3 | 1.44 |
| A840V | A840V_GCG_GTA | 5 | 7 | 7 | 1.34 |
| A840G | A840G_GCG_GGG | 6335 | 8469 | 8124 | 1.28 |
| A840H | A840H GCG CAC | 1 | 88 | 15 | 0.16 |
| A840H | A840H_GCG_CAT | 1 | 910940 | 230659 | 0.24 |

TABLE 3

| | Sequence | |
|---|---|---|
| Exemplary Cas9 Type | Identifier | Sequence |
| Cas9 nickase (H840A), | SEQ ID NO: | MDKKYSIGLDIGTNSVGWAVITDEYKVPSK |
| Streptococcus pyogenes | 22 | KFKVLGNTDRHSIKKNLIGALLFDSGETAE |
| | | ATRLKRTARRRYTRRKNRICYLQEIFSNEM |
| | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGNL |
| | | IALSLGLTPNFKSNFDLAEDAKLQLSKDTY |
| | | DDDLDNLLAQIGDQYADLFLAAKNLSDAIL |
| | | LSDILRVNTEITKAPLSASMIKRYDEHHQDL |
| | | TLLKALVRQQLPEKYKEIFFDQSKNGYAGY |
| | | IDGGASQEEFYKFIKPILEKMDGTEELLVKL |
| | | NREDLLRKQRTFDNGSIPHQIHLGELHAILR |
| | | RQEDFYPFLKDNREKIEKILTFRIPYYVGPL |
| | | ARGNSRFAWMTRKSEETITPWNFEEVVDK |
| | | GASAQSFIERMTNFDKNLPNEKVLPKHSLL |
| | | YEYFTVYNELTKVKYVTEGMRKPAFLSGE |
| | | QKKAIVDLLFKTNRKVTVKQLKEDYFKKI |
| | | ECFDSVEISGVEDRFNASLGTYHDLLKIIKD |
| | | KDFLDNEENEDILEDIVLTLTLFEDREMIEE |
| | | RLKTYAHLFDDKVMKQLKRRRYTGWGRL |
| | | SRKLINGIRDKQSGKTILDFLKSDGFANRNF |
| | | MQLIHDDSLTFKEDIQKAQVSGQGDSLHEH |
| | | IANLAGSPAIKKGILQTVKVVDELVKVMGR |
| | | HKPENIVIEMARENQTTQKGQKNSRERMK |
| | | RIEEGIKELGSQILKEHPVENTQLQNEKLYL |
| | | YYLQNGRDMYVDQELDINRLSDYDVDAIV |
| | | PQSFLKDDSIDNKVLTRSDKNRGKSDNVPS |
| | | EEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLN |
| | | AVVGTALIKKYPKLESEFVYGDYKVYDVR |
| | | KMIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTV |
| | | AYSVLVVAKVEKGKSKKLKSVKELLGITIM |
| | | ERSSFEKNPIDFLEAKGYKEVKKDLIIKLPK |
| | | YSLFELENGRKRMLASAGELQKGNELALP |
| | | SKYVNFLYLASHYEKLKGSPEDNEQKQLF |
| | | VEQHKHYLDEIIEQISEFSKRVILADANLDK |
| | | VLSAYNKHRDKPIREQAENIIHLFTLTNLGA |
| | | PAAFKYFDTTIDRKRYTSTKEVLDATLIHQS |
| | | ITGLYETRIDLSQLGGD |
| Cas9 Wild-Type, | SEQ ID NO: | MDKKYSIGLDIGTNSVGWAVITDEYKVPSK |
| Streptococcus pyogenes | 23 | KFKVLGNTDRHSIKKNLIGALLFDSGETAE |
| | | ATRLKRTARRRYTRRKNRICYLQEIFSNEM |
| | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGNL |
| | | IALSLGLTPNFKSNFDLAEDAKLQLSKDTY |
| | | DDDLDNLLAQIGDQYADLFLAAKNLSDAIL |
| | | LSDILRVNTEITKAPLSASMIKRYDEHHQDL |
| | | TLLKALVRQQLPEKYKEIFFDQSKNGYAGY |
| | | IDGGASQEEFYKFIKPILEKMDGTEELLVKL |
| | | NREDLLRKQRTFDNGSIPHQIHLGELHAILR |
| | | RQEDFYPFLKDNREKIEKILTFRIPYYVGPL |
| | | ARGNSRFAWMTRKSEETITPWNFEEVVDK |
| | | GASAQSFIERMTNFDKNLPNEKVLPKHSLL |
| | | YEYFTVYNELTKVKYVTEGMRKPAFLSGE |
| | | QKKAIVDLLFKTNRKVTVKQLKEDYFKKI |
| | | ECFDSVEISGVEDRFNASLGTYHDLLKIIKD |
| | | KDFLDNEENEDILEDIVLTLTLFEDREMIEE |
| | | RLKTYAHLFDDKVMKQLKRRRYTGWGRL |
| | | SRKLINGIRDKQSGKTILDFLKSDGFANRNF |
| | | MQLIHDDSLTFKEDIQKAQVSGQGDSLHEH |
| | | IANLAGSPAIKKGILQTVKVVDELVKVMGR |
| | | HKPENIVIEMARENQTTQKGQKNSRERMK |
| | | RIEEGIKELGSQILKEHPVENTQLQNEKLYL |
| | | YYLQNGRDMYVDQELDINRLSDYDVDHIV |
| | | PQSFLKDDSIDNKVLTRSDKNRGKSDNVPS |
| | | EEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLN |
| | | AVVGTALIKKYPKLESEFVYGDYKVYDVR |
| | | KMIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTV |
| | | AYSVLVVAKVEKGKSKKLKSVKELLGITIM |
| | | ERSSFEKNPIDFLEAKGYKEVKKDLIIKLPK |
| | | YSLFELENGRKRMLASAGELQKGNELALP |
| | | SKYVNFLYLASHYEKLKGSPEDNEQKQLF |
| | | VEQHKHYLDEIIEQISEFSKRVILADANLDK |
| | | VLSAYNKHRDKPIREQAENIIHLFTLTNLGA |
| | | PAAFKYFDTTIDRKRYTSTKEVLDATLIHQS |
| | | ITGLYETRIDLSQLGGD |

While the invention has been described in detail and with reference to specific examples thereof, it will be apparent to one skilled in the art that various changes and modifications can be made therein without departing from the spirit and scope thereof.

### REFERENCES

1. Jinek, M., et al., A programmable dual-RNA-guided DNA endonuclease in adaptive bacterial immunity. Science, 2012. 337 (6096): p. 816-21.
2. Komor, A. C., et al., Programmable editing of a target base in genomic DNA without double-stranded DNA cleavage. Nature, 2016. 533 (7603): p. 420-4.
3. Gaudelli, N. M., et al., Programmable base editing of A*T to G*C in genomic DNA without DNA cleavage. Nature, 2017. 551 (7681): p. 464-471.
4. Anzalone, A. V., et al., Search-and-replace genome editing without double-strand breaks or donor DNA. Nature, 2019. 576 (7785): p. 149-157.
5. Roy, S., et al., Cas9/Nickase-induced allelic conversion by homologous chromosome-templated repair in Drosophila somatic cells. Sci Adv, 2022. 8 (26): p. eabo0721.
6. Tong, Y., et al., A versatile genetic engineering toolkit for E. coli based on CRISPR-prime editing. Nat Commun, 2021. 12 (1): p. 5206.
7. Chen, S., et al., fastp: an ultra-fast all-in-one FASTQ preprocessor. Bioinformatics, 2018. 34 (17): p. 884-890.
8. Martin M. Cutadapt removes adapter sequences from high-throughput sequencing reads. EMBnet. journal. 2011. 17 (1): p. 10-2.

1. A modified Cas9 protein, comprising one or more mutations listed in Table 2.

2. The modified Cas9 protein of claim 1, comprising one or more mutations selected from the group consisting of I830S, I830A, I830G, I830L, I830F, N831A, N831L, N831S, N831K, R832E, R832D, R832H, R832L, R832Q, R832V, L833Q, L833S, L833T, S834P, S834R, V842I, P843T, Q844R, F846Y, D850V, D850E, D850H, and D850Y relative to the wild-type Cas9 amino acid sequence of SEQ ID NO: 23.

3. The modified Cas9 protein of claim 1, wherein the modified Cas9 protein comprises one or more mutations selected from the group consisting of R832E, R832D, R832H, R832L, I830S, N831A, R832Q, D850Y, D850V, D850E, and F846Y relative to the wild-type Cas9 amino acid sequence of SEQ ID NO: 23.

4. The modified Cas9 protein of claim 1, wherein the modified Cas9 protein comprises one or more mutations selected from the group consisting of R832E, R832E, D850Y, and D850V relative to the wild-type Cas9 amino acid sequence of SEQ ID NO: 23.

5. The modified Cas9 protein of claim 1, comprising one or more mutations selected from the group consisting of A840Y, A840L, A840F, A840W, A840M, A840C, A840E, A840R, A840S, A840N, A840T, A840D, A840Q, A840K, A840V, A840I, A840G, and A840P relative to a control Cas9 nickase amino acid sequence of SEQ ID NO: 22.

6. The modified Cas9 protein of claim 1, wherein the modified Cas9 protein comprises one or more mutations selected from the group consisting of A840Y, A840L, A840F, A840W, A840M, A840C, A840E, A840R, A840S, A840N, and A840T relative to a control Cas9 nickase amino acid sequence of SEQ ID NO: 22.

7. The modified Cas9 protein of claim 1, wherein the modified Cas9 protein comprises one or more mutations selected from the group consisting of A840Y, A840L, A840F, A840W, and A840M relative to a control Cas9 nickase amino acid sequence of SEQ ID NO: 22

8. The modified Cas9 protein of claim 1, further comprising at least one nuclear localization signal.

9. The modified Cas9 protein of claim 1, wherein the modified Cas9 protein exhibits enhanced nickase activity relative to the wild-type Cas9 having the sequence of SEQ ID NO: 23 or the control Cas9 nickase having the sequence of SEQ ID NO: 22.

10. The modified Cas9 protein of claim 1, wherein the modified Cas9 protein comprises one or more mutations that confer increased nickase activity relative to the control Cas9 nickase having the sequence of SEQ ID NO: 22.

11. The modified Cas9 protein of claim 1, wherein the modified Cas9 protein binds to a pegRNA and exhibits prime editing activity enhanced by at least an amount selected from the group consisting of 5%, 10%, 15%, 20%, 25%, and 30%, relative to the control Cas9 nickase having the sequence of SEQ ID NO: 22.

12. An isolated nucleic acid encoding the modified Cas9 protein of claim 1.

13. An mRNA encoding the modified Cas9 protein of claim 1.

14. A method for isolating the modified Cas9 protein of claim 1, the method comprising:
(a) generating a library of Cas9 protein variants through saturation mutagenesis of one or more target codons within a nucleic acid sequence encoding Cas9 protein;
(b) cloning the mutated nucleic acid encoding Cas9 protein along with a nucleic acid encoding a pegRNA which targets an antibiotic resistance gene into the same or different plasmid vector;
(c) introducing the plasmid vector(s) into a host cell population with a separate plasmid encoding a deactivated antibiotic resistance gene;
(d) subjecting the host cell population to antibiotic selection; and
(e) isolating modified Cas9 protein variants that exhibit enhanced prime editing activity with pegRNA to restore antibiotic resistance,
thereby isolating a modified Cas9 protein that functions with a prime editing guide RNA (pegRNA) that exhibits enhanced prime editing activity relative to the wild-type Cas9 having the sequence of SEQ ID NO: 23 or the control Cas9 nickase having the sequence of SEQ ID NO: 22.

15. The method of claim 14, wherein the target codons within the nucleic acid sequence encoding Cas9 nickase encode amino acids at a position selected from the group consisting of 830, 831, 832, 833, 834, 835, 836, 837, 838, 839, 840, 841, 842, 843, 844, 845, 846, 847, 848, 849 and 850.

16. The method of claim 1, wherein the host cell is selected from the group consisting of bacterial cells, insect cells, or mammalian cells.

17. The method of claim 1, wherein the modified Cas9 nickase that functions with the pegRNA exhibits prime editing activity enhanced by at least an amount selected from the group consisting of 5%, 10%, 15%, 20%, 25%, and 30%, relative to the wild-type Cas9 having the sequence of SEQ ID NO: 23 or the control Cas9 nickase having the sequence of SEQ ID NO: 22.

18. The method of claim 1, wherein the saturation mutagenesis targets specific codons within a Cas9 nickase domain selected from the group consisting of the REC Lobe; the Bridge Helix; the NUC Lobe; the RuvC Domain; the HNH Domain; the PAM-Interacting Domain; and the C Terminal Domain.

19. The method of claim 1, wherein the mutations are located in a Cas9 nickase domain selected from the group consisting of REC1, REC2, REC3, and combinations thereof.

20. The method of claim 1, wherein the pegRNA targets a kanamycin resistance gene.

21-32. (canceled)


SEQUENCE LISTING (23 sequences)

SEQ ID NO: 1 - DNA, 53 bp, synthetic construct
  source 1..53: mol_type = other DNA
tatgtacgtggaccaagagttggatnnkaatcggctgagtgactacgacgttg

SEQ ID NO: 2 - DNA, 53 bp, synthetic construct
  source 1..53: mol_type = other DNA
gtacgtggaccaagagttggatattnnkcggctgagtgactacgacgttgatc

SEQ ID NO: 3 - DNA, 53 bp, synthetic construct
  source 1..53: mol_type = other DNA
cgtggaccaagagttggatattaatnnkctgagtgactacgacgttgatcata

SEQ ID NO: 4 - DNA, 53 bp, synthetic construct
  source 1..53: mol_type = other DNA
ggaccaagagttggatattaatcggnnkagtgactacgacgttgatcatatcg

SEQ ID NO: 5 - DNA, 53 bp, synthetic construct
  source 1..53: mol_type = other DNA
ccaagagttggatattaatcggctgnnkgactacgacgttgatcatatcgtcc

SEQ ID NO: 6 - DNA, 53 bp, synthetic construct
  source 1..53: mol_type = other DNA
agagttggatattaatcggctgagtnnktacgacgttgatcatatcgtcccgc

SEQ ID NO: 7 - DNA, 53 bp, synthetic construct
  source 1..53: mol_type = other DNA
gttggatattaatcggctgagtgacnnkgacgttgatcatatcgtcccgcaga

SEQ ID NO: 8 - DNA, 53 bp, synthetic construct
  source 1..53: mol_type = other DNA
ggatattaatcggctgagtgactacnnkgttgatcatatcgtcccgcagagct

SEQ ID NO: 9 - DNA, 53 bp, synthetic construct
  source 1..53: mol_type = other DNA
tattaatcggctgagtgactacgacnnkgatcatatcgtcccgcagagcttcc

SEQ ID NO: 10 - DNA, 53 bp, synthetic construct
  source 1..53: mol_type = other DNA
taatcggctgagtgactacgacgttnnkcatatcgtcccgcagagcttcctca

SEQ ID NO: 11 - DNA, 53 bp, synthetic construct
  source 1..53: mol_type = other DNA
tcggctgagtgactacgacgttgatnnkatcgtcccgcagagcttcctcaaag

SEQ ID NO: 12 - DNA, 53 bp, synthetic construct
  source 1..53: mol_type = other DNA
gctgagtgactacgacgttgatcatnnkgtcccgcagagcttcctcaaagacg

SEQ ID NO: 13 - DNA, 53 bp, synthetic construct
  source 1..53: mol_type = other DNA
gagtgactacgacgttgatcatatcnnkccgcagagcttcctcaaagacgatt

SEQ ID NO: 14 - DNA, 53 bp, synthetic construct
  source 1..53: mol_type = other DNA
tgactacgacgttgatcatatcgtcnnkcagagcttcctcaaagacgattcta

SEQ ID NO: 15 - DNA, 53 bp, synthetic construct
  source 1..53: mol_type = other DNA
ctacgacgttgatcatatcgtcccgnnkagcttcctcaaagacgattctattg

SEQ ID NO: 16 - DNA, 53 bp, synthetic construct
  source 1..53: mol_type = other DNA
cgacgttgatcatatcgtcccgcagnnkttcctcaaagacgattctattgaca

SEQ ID NO: 17 - DNA, 53 bp, synthetic construct
  source 1..53: mol_type = other DNA
cgttgatcatatcgtcccgcagagcnnkctcaaagacgattctattgacaata

SEQ ID NO: 18 - DNA, 53 bp, synthetic construct
  source 1..53: mol_type = other DNA
tgatcatatcgtcccgcagagcttcnnkaaagacgattctattgacaataagg

SEQ ID NO: 19 - DNA, 53 bp, synthetic construct
  source 1..53: mol_type = other DNA
tcatatcgtcccgcagagcttcctcnnkgacgattctattgacaataaggtac

SEQ ID NO: 20 - DNA, 53 bp, synthetic construct
  source 1..53: mol_type = other DNA
tatcgtcccgcagagcttcctcaaannkgattctattgacaataaggtactga

SEQ ID NO: 21 - DNA, 53 bp, synthetic construct
  source 1..53: mol_type = other DNA
cgtcccgcagagcttcctcaaagacnnktctattgacaataaggtactgacgc

SEQ ID NO: 22 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - protein, 1368 aa, Streptococcus pyogenes [sequence omitted]